(19) United States
(12) Patent Application Publication
(10) Pub. No.: US 2002/0076404 A1
(43) Pub. Date: Jun. 20, 2002

(54) Treating atopic dermatitis with IgE antagonists

(76) Inventor: Tse Wen Chang, Hsinchu (TW)

Correspondence Address: TANOX, INC., 10301 STELLA LINK, HOUSTON, TX, 77025, US

(21) Appl. No.: 09/991,224

(22) Filed: Nov. 19, 2001

Related U.S. Application Data
(63) Continuation of application No. 09/240,476, filed on Jan. 29, 1999, now abandoned.
(60) Provisional application No. 60/073,033, filed on Jan. 29, 1998.

(51) Int. Cl. A61K 39/395

(52) U.S. Cl. 424/131.1; 424/142.1

(57) ABSTRACT

The invention relates to a composition for treatment of atopic dermatitis comprising a suitable IgE antagonist that does not induce the release of mediators of allergy; for example, anti-IgE antibodies that bind to secreted IgE, membrane IgE on the surface of IgE-producing B cells, but not to IgE bound to the FcεRI on the surface of basophils or mast cells. Preferably, these antibodies also do not bind to IgE bound to FcεRII receptors. It is also preferable if these antibodies have human IgG1 or IgG3 constant regions, as well as further human portions, if desired. The composition can be administered systemically or topically.

This application claims priority to U.S. provisional application No. 60/073,033 filed Jan. 29, 1998, and to U.S. nonprovisional application No. 09/240,476 filed Jan. 29, 1999.

### BACKGROUND OF THE INVENTION

Immunoglobuin E (IgE) is one class of immunoglobulin (or “antibody”) molecules. IgE is present in humans in lower concentrations than the other immunoglobuins: IgG, IgM, IgA, and IgD. IgE is thought to have a role in protection against parasites, but has never been definitively established as playing a necessary, or even a beneficial role, at least in developed countries, where parasite infections are not a significant problem. IgE is well known, however, as the mediator of immediate-type hypersensitivity allergic reactions, including allergic rhinitis (“hay fever”), extrinsic asthma, and food and drug allergies.

In IgE-mediated allergic reactions, IgE, after it is secreted by B cells, binds, through its Fc portion to the FcεRI receptors, which are present on the surface of basophils, mast cells and Langerhans cells. If the IgE bound to the surface of these cells now contacts and binds an allergen, this causes a cross-linking of the bound IgE molecules and hence the underlying receptors, and triggers the release of pharmacologic mediators, such as histamine, serotonin, leukotrines and the slow-reacting substance of anaphylaxis. These mediators cause the pathologic manifestations of allergic reactions.

Some patients with a history of some or all of the IgE-mediated allergic conditions also suffer from a painful skin condition called atopic dermatitis. Atopic dermatitis is characterized by pruritis (itching), redness, and painful skin lesions. In some patients with chronic dermatitis, the skin can become lichenified. While atopic dermatitis is usually found together with other allergic diseases, no correlation has been established between high IgE levels and the severity, or the manifestation, of atopic dermatitis in affected patients. Those in the field believe that atopic dermatitis may have a link to IgE, but that IgE alone is not the causative agent.

A particular class of anti-IgE antibodies has been developed to treat allergic diseases. These antibodies bind to secreted IgE, but not to IgE attached to the FcεRI receptors. When these anti-IgE antibodies are administered internally, they bind to IgE and netralize it. preventing its binding to either FcεRI or FcεRII receptors, the latter being present on B cells and other cell types as well. These anti-IgE antibodies also bind to IgE which is attached to the membrane of IgE-producing B cells (the “membrane form of IgE”). By doing so, they may further aid in down-regulating or eliminating, through antibody dependent cellular cytotoxicity (“ADCC”) or complement mediated cytolysis, the IgE-producing B cells, and therefore, reduce the levels of secreted IgE. Because they do not bind to IgE attached to the FcεRI, however, they do not cause cross-linking and do not themselves result in release of pharmacologic mediators of allergy.

It has been shown that such anti-IgE antibodies can lower IgE levels, in both animal models and human clinical trials. See Come et al., J. Clin. Invest. 99, No. 5, 879-887 (1997). Such anti-IgE antibodies also demonstrated efficacy in treating allergic rhinitis and extrinsic asthma in several human clinical trials, as would be expected from the fact that they lower IgE levels. See Come et al., id.; Boulet et al., Am J. Respir. Crit. Care Med., 155: 1835-1840 (1997); Fahy et al., J. Respir. Crit. Care Med., 155: 1828-1834 (1997). No clinical trials of these anti-IgE antibodies have been performed for treatment of atopic dermatitis. Based on the lack of correlation between high IgE levels and severity of atopic dermatitis symptoms, it would not be expected that these antibodies would be useful in atopic dermatitis treatment. Regarding other IgE antagonists which function by preventing or inhibiting IgE from binding to the FcεRI without inducing release of the pharmacologic mediators of allergy, they would also not be expected to be an effective atopic dermatitis treatment. Such antagonists would include small molecules and other new chemical or biological entities.

### SUMMARY OF THE INVENTION

The invention includes a composition for treating atopic dermatitis comprising IgE antagonists which do not induce release of the pharmacologic mediators of allergy. Such antagonists include monoclonal anti-IgE antibodies which bind to secreted IgE but not to the FcεRI receptors present on basophils, mast cells, or Langerhans cells. The anti-IgE antibodies preferably also bind to membrane IgE, and thereby aid in down-regulating or eliminating IgE-producing B cells, leading to further reduction in secreted IgE levels. These anti-IgE antibodies preferably do not bind to the low affinity FcεRII receptors. If the antibodies of the invention did bind to IgE bound to the FcεRII receptors, they could cause the destruction or down-regulation of B cells producing other classes of immunoglobullins, or other cell types, which would be undesirable.

Notwithstanding the conventional wisdom that depleting or removing IgE, without more, will not be effective in treatment of atopic dermatitis, this patent application rests on the premise that IgE antagonists including anti-IgE alone, will prove an effective treatment. In fact, anti-IgE should prove to be a substantial benefit to patients with atopic dermatitis, well beyond the expectations of those in the field.

If the IgE antagonist chosen is anti-IgE, they can be modified in order to make them less antigenic and more suitable for human administration by techniques including chimerization, humanization (through CDR-grafting), or otherwise, including producing fully human antibodies in transgenic animals or producing fragments of human antibodies through phage display library technology. Preferably, the antibodies have a human IgG1 or IgG3 constant heavy chain region, as such regions are known to mediate ADCC and complement mediated cytolysis, thereby aiding in elimination of IgE-producing B cells.

The pharmaceutical composition of the invention is likely to be most effective when internally administered, such as by intravenous, intramuscular, or subcutaneous injection. It could also be internally administered by oral ingestion, in a suitable carrier which is not subject to digestive degradation, or through the alveoli of the lung by an inhaler. It may also be possible to administer the composition topically to affected areas, where it would be absorbed and act locally.

### DESCRIPTION OF MAKING AND USING THE INVENTION

### 1. Making the Various Embodiments of the Invention

Chemical or biological entities suitable for use as IgE antagonists can be selected an screened by a number of methods, including using assays similar to those used to screen TES-C21, described below. In esssence, one would screen first for those that bound to secreted IgE, and then, from that group, those that did not induce release of pharmacologic mediators of allergy would be selected. A number of different assays, well known to those in the art, could be used to accomplish this.

In one specific embodiment, the monoclonal anti-IgE antibodies used with this invention are produced by continuous (immortalized), stable, antibody-producing cell lines. The preferred antibody-producing cell lines are hybridoma and transfectoma cell lines. However, they can be any cell lines which contain and are capable of expressing functionally rearranged genes which encode the antibodies (or fragments) of interest. Lymphoid cells which naturally produce assembled immunoglobulin are preferred.

Hybridoma cells which produce the specific antibodies used with this invention can be made by the standard somatic cell hybridization technique of Kohler and Milstein, Nature 256:495 (1975) or similar procedures employing different fusing agents. Briefly, the procedure is as follows. The monoclonal anti-IgE antibodies are produced by immunizing an animal with human IgE or IgE-producing B cells, or peptidic segments of human IgE (secretory or membrane), which are identified as including the epitope of interest, which is in the Fc region of IgE. Peptides can be synthesized or produced by recombinant DNA technology and, for enhanced antigenic effect, conjugated to a carrier protein, such as keyhole limpet hemocyanin. Following immunization, lymphoid cells (e.g., splenic lymphocytes) are obtained from the immunized animal and fused with immortalizing cells (e.g., myeloma or heteromyeloma) to produce hybrid cells. The hybrid cells are screened to identify those which produce the desired anti-IgE antibody by following the screening methods described below in detail.

It is preferred that, when long-term administration of antibodies is contemplated as it is here where anti-IgE is used for treating atopic dermatitis, the antibodies be either human or substantially human, to reduce or eliminate the human anti-mouse (HAMA) response. The murine antibody portions could themselves trigger an allergic response, or the HAMA response against such portions could reduce the effectiveness of the treatment.

Human hybridomas which secrete human antibodies can be produced by the Köhler and Milstein technique. Although human antibodies are especially preferred for treatment of humans, in general, the generation of stable human-human hybridomas by such techniques for long-term production of human monoclonal antibody can be difficult. An alternative technique for producing human antibodies is production in transgenic mice. Briefly, this approach involves disruption of endogenous murine heavy and kappa light chain loci, followed by construction of heavy and κ light chain transgenes containing V, D, J segments, and C genes of human origin. These are then introduced by pronuclear microinjection using human transgenes. The mice are then cross-bred to generate the human antibody producing strains. This technique is describe in more detail in, among other references, U.S. Pat. No. 5,569,825. The technology may be available under license from GenPharm International, Inc. (Mountain View, Calif.).

Another alternative for solving antigenicity problems is to produce fully human antibody fragments, for example, the single chain Fv region, by the phage display library methodology. Briefly, this involves amplification of the human V gene repertoire from bone marrow, blood and tonsil samples by polymerase chain reaction (“PCR”), followed by preparation of separate libraries containing heavy and light chain (both κ and λ) chain V genes. These separate fragments are then assembled into a single chain Fv for display on the surface of phage, where the desired fragments can be readily screened. References describing this technique in more detail include U.S. Pat. No.5,565,332 and European Patent No.0 589 877 B1. The technology may also be available under license from Cambridge Antibody Technology Limited, Melbourn, England.

Production of antibodies in rodents, especially mice, is a very well established procedure. An alternative to reduce the murine portions of the anti-IgE antibodies is to produce them in a rodent system, and convert them into chimeric rodent/human antibodies or CDR-grafted humanized antibodies by established techniques. Chimeric antibodies can be produced as described, for example, in U.S. Pat. No. 4,816,397. The making of humanized antibodies is described, among other sources, in U.S. Pat. Nos. 5,693,762; 5,693,761; 5,225,539, and in WO 89/06692 and WO 92/22653.

One example of an anti-IgE antibody of the invention (designated TES-C21) and its chimeric mouse-human form (designated TESC-2) is described in International Application WO 92/17207. The screening protocols (described below) for TES-C21 and TESC-2 can be applied to other anti-IgE antibodies to yield antibodies of the invention suitable for chimerization or humanization through CDR-grafting. The hybridoma cell lines producing TES-C21 are available from the American Type Culture Collection (“ATCC”), Rockville, Md. under Accession No.11134, and those producing TESC-2 are on deposit under Accession No. BRL 10706.

A humanized version of the murine antibody TES-C21 was made, as described in detail in Australian Patent No. 675449, granted May 25,1997. Similar procedures can be followed to produce other humanized anti-IgE antibodies. Several transfectomas producing humanized anti-IgE antibodies suitable for use with the invention are available from the ATCC under the following accession numbers: 11130; 11131; 11132; 11133. An anti-IgE antibody similar to that produced from the transfectoma deposited under accession number 11131 is among those with potential for full clinical development for treatment of atopic dermatitis. Another humanized antibody suitable for treatment of atopic dermatitis is E25 (rhuMAb-E25), produced by Genentech, Inc. This antibody is described in Presta et al., J. Immunol. 151:2623-2632 (1993).

### A. Production and Screening of TES-C21 and TESC-2

TES-C21 and TESC-2 were produced and screened as follows. Briefly, male Balb/c mice were immunized several times with polyclonal human IgE from sera (provided by Ventrex). The IgE was combined with a suitable adjuvant. Mice were sacrificed after the last injection of immunogen and the spleens were removed for preparing single cell suspensions for fusion with myeloma cells. The spleen cells were fused with Sp2/0 cells using a fusion mixture of polyethylene glycol 1450 (Kodak), CMF-PBS and DMSO. DMEM was added after the cell suspensions were combined.

The hybridomas resulting from the fusion were then screened by enzyme-linked immunosorbent assay (ELISA) against human IgE bound to an Immulon 2 plate. One of these hybridomas produced TES-C21.

Sp 2/0 cells were co-transfected with the variable regions of TES-C21 H and L-chains, and human γ1 and κ constant regions, and aliquoted into 96 well plates for selection. Supernatants were screened for secretion of human IgG which bound to human IgE.

The transfectoma cells were adapted to growth in serum-free medium. TESC-2 was then purified from medium of confluent cultures using an immobilized protein A column.

TES-C21 was further screened, by ELISA, to be specific for human IgE, and not to react with IgG, IgM, IgA, IgD, human serum albumin, transferrin or insulin. TES-C21 bound equally well to various human IgE molecules. TES-C21 bound to the IgE-secreting cell lines SKO-007, U266 and SE44 in a dose-dependent manner, indicating binding to human membrane IgE. But TES-C21 did not bind to human B cell lines bearing surface IgM, IgD, IgG, or IgA, or to a T cell line, or to the parent murine cell line of SE44, or to a murine cell line secreting chimeric human IgG. TES-C21 also does not bind to IgE on low affinity FcεRII receptors which are present on a wide variety of cell types. It also did not induce histamine release from freshly prepared human blood basophils, on which the FcεR are armed with IgE. At 10 μg/ml TES-C21 is able to inhibit completely the binding of 1 μg of IgE to FcεRII.

TESC-2 and TES-C21 bind equally well to IgE bound to microtiter plates. This was demonstrated as follows. Immulon 2 plates were coated with gp120 peptide-ovalbumin conjugate and IgE-SE44 was bound to the immobilized antigen. TES-C21 or TESC-2 at various concentrations were added. Binding was detected using either horseradish peroxidase (“HRP”)-conjugated goat antimouse IgG (for TES-C21) or HRP-goat antihuman IgG, Fc (for TESC-2).

It was determined that TESC-2 and TES-C21 also have the same relative affinity for IgE bound to microtiter plates. Immulon 2 plates were coated with gp120 peptide-ovalbumin conjugate and IgE-SE44 was bound to the immobilized antigen. TES-C21 and TESC-2 at various concentrations were added and preincubated for 1 hour before adding 0.22 μg/ml of biotinylated TES-C21. Binding of biotinylated TES-C21 was detected using horseradish peroxidase-conjugated streptavidin.

TESC-2 and TES-C21 also were shown to bind equally to IgE-producing cells. This was demonstrated by incubating such cells at 2×10⁶ cells/100 μl PBS-1% goat serum at various antibody concentrations at 0° for 30 minutes. Binding of TES-C21 was detected using FITC-goat (Fab)₂ antimouse IgG; binding of TESC-2 was detected using FITC-goat (Fab)₂ antihuman IgG. Binding was quantitated by fluorescence flow cytometry using a Coulter Epics V. The FITC intensity gate was set to yield 10%±0.5% positive cells in the absence of primary immunoglobulins.

It was found that neither TES-C21 nor TESC-2 bound to IgE which was bound to low affinity IgE receptors. The possibility that TESC-2 recognized IgE complexed with CD23 was studied using cells of an IgG-secreting human lymphoblastoid line, IM-9. The presence of CD23 on IM-9 cells was confirmed by their strong staining with anti-Leu 20, a MAb specific for CD23. IM-9 cells were incubated with 5 to 10 μg/ml of human IgE, washed, and then incubated with biotin-labeled TESC-2 or a positive control anti-IgE MAb TES-19, followed by FITC-streptavidin and analyzed by flow cytometry.

Both chimeric TESC-2 and murine TES-C21 were shown to inhibit binding of IgE to FcεRII. The antibodies were preincubated at various concentrations with 20 μg IgE-SE44 for 1 hour at 37° before addition of IM-9 cells bearing FcεRII. Binding to IgE was detected using biotinylated TES-19 and FITC-streptavidin and quantitated by fluorescence flow cytometry.

To negate the possibility that immune complexes of TESC-2 and IgE were formed during their preincubation in these experiments, thereby yielding false positives, it was confirmed that these immune complexes also did not bind to FcεRII, using biotin-labeled TESC-2 or FITC goat anti-human IgE (with TES-C21).

As shown in Table 1 below, neither TESC-2 nor TES-C21 induces histamine release from freshly prepared human blood basophils, on which the FcεR are armed with IgE. Due to the variable release of mediators from basophils of different donors, the antibodies were examined at multiple concentrations on basophil preparations from multiple donors. No induction of histamine release by TESC-2 or TES-C21 was observed.

TABLE 1

| | Concentration | Net Histamine Release | | | |
|---|---|---|---|---|---|
| Antibody | μg/ml | Donor 1 | Donor 2 | Donor 3 | Donor 4 |
| Polyclonal | 0.1 | 70 | 64 | 55 | 81 |
| goat anti-IgE | | | | | |
| TESC-2 | 0.4 | 0 | 2 | | |
| | 2 | 0 | 3 | | |
| | 10 | 0 | 2 | | |
| | 50 | 0 | 2 | | |
| TES-C21 | 0.4 | | | 1 | 0 |
| | 2 | | | 1 | 0 |
| | 10 | | | 0 | 0 |
| | 50 | | | 1 | 0 |

To address the possibility that TES-C21 might bind to basophils and induce cross-linking of the receptors to induce histamine release, upon introduction of a cross-linking antigen or other agent, a secondary antibody was used for crosslinking. Since anti-human IgG alone can induce histamine release, only the murine antibody TES-C21 was used in these experiments. The crosslinking goat antimouse IgG enhances histamine release induced by suboptimal concentrations of control anti-IgEs. However, TES-C21 did not induce histamine under these very permissive conditions.

TESC-2 was further tested to determine whether it could block the binding of IgE to FcεRI receptors, and whether immune complexes of IgE and TESC-2 would bind to these receptors. To determine whether TESC-2 inhibits the binding of human IgE to FcεRI, human peripheral blood basophils that had been depleted of IgE by treatment at low pH were reloaded or sensitized with SE44-derived chimeric IgE reactive to a peptide antigen. Functional binding of SE44 IgE was tested by histamine release induced by the polyvalent R15K peptide-ovalbumin conjugate to which the variable region of IgE-SE44 binds. Preincubation of IgE-SE44 with TESC-2 inhibited IgE binding to FcεRI (Table 2). Binding of SE44 IgE was also inhibited when basophils were incubated with another IgE (PS) before exposure to IgE-SE44. It may be assumed that immune complexes of TESC-2 and IgE were formed during the preincubation and did not cause the release of histamine. The experimental conditions and the results of these experiments are summarized below in Table 2.

TABLE 2
Inhibition of IgE Binding to High-Affinity
IgE Receptors by TESC-2

| | Net Histamine Release (% of total) | |
|---|---|---|
| Conditions for Basophil | Challenge with R15K | Challenge with |
| Loading with IgE-SE44 | Peptide-Ovalbumin | Anti-IgE |
| IgE-SE44 was not preincubated | 37 | 66 |
| with TESC-2 | | |
| IgE-SE44 was preincubated with | 3 | 68 |
| TESC-2 | | |
| IgE-SE44 was preincubated with | 0 | 63 |
| IgE-PS | | |

These studies have also been performed, and similar results obtained, with the CDR-grafted versions of TES-C21 referenced above.

### 2. Using the Antibodies of the Invention for Treatment of Atopic Dermatitis

Prior to commercial availability, the IgE antagonists, or antibodies, of the invention must be subjected to human clinical trials to confirm their safety and efficacy. A sample protocol for such a clinical trial would be to start with up to 30 patients affected by atopic dermatitis who continue to have symptoms despite regular medical care for atopic dermatitis, including such treatments as anti-histamines, corticosteroids, soothing baths and lotions. Patients would receive intravenous or subcutaneous injections of 50 to 300 mg of anti-IgE at weekly, bi-weekly or monthly intervals for 3 to 6 months. Effects of anti-Ige treatment on their ongoing atopic dermatitis would be scored, e.g., using the SCORAD index (see B. Kung et al., Dermatology 1997, 195:10-19). The relationship between effect on circulating IgE concentration and clinical efficacy could be assessed.

Additional studies would investigate alternative dosing schedules and dosing intervals and compare anti-IgE treatment to placebo formulations. The efficacy of topically applied IgE would also be studied.

The composition of the invention, administered by any acceptable route, is expected to have a substantial beneficial effect for patients sufferring from atopic dermatitis.

The foregoing description, terms, expressions and examples are exemplary only and not limiting. The invention includes all equivalents of the foregoing embodiments, both known and unknown. The invention is limited only by the claims which follow and not by any statement in any other portion of this document or in any other source.

What is claimed is:

1. A method for treating atopic dermatitis comprising administering to a host in need of such treatment a composition comprising an IgE antagonist.

2. A method for treating atopic dermatitis comprising administering to a host in need of such treatment a composition comprising an anti-IgE antibody which binds to secreted IgE but not to basophils.

3. A method for treating atopic dermatitis comprising administering to a host in need of such treatment a composition comprising an anti-IgE antibody, which binds to secreted IgE and membrane-bound IgE, but not to basophils.

4. A method for treating atopic dermatitis comprising administering to a host in need of such treatment a composition comprising an anti-IgE antibody which binds to secreted IgE and membrane-bound IgE but not to basophils and not to IgE which is bound to the FcεRII receptor.

5. The method of any of claims 2 to 4, wherein the anti-IgE antibody does not bind to mast cells.

6. The method of any of claims 2 to 4, wherein the anti-IgE antibody is a monoclonal antibody.

7. The method of claim 6, wherein the anti-IgE antibody is a chimeric, humanized (CDR-grafted), or human antibody.

8. The method of claim 6, wherein the anti-IgE antibody targets human IgE.

9. The method of claim 8, wherein the anti-IgE antibody has a human IgG1 or IgG3 heavy chain constant region.

10. The method of any of claims 2 to 9, wherein the composition further comprises a pharmacologically acceptable carrier, excipient, stabilizer, and/or diluent.

11. The method of claim 10, wherein the composition is suitable for subcutaneous or intravenous injection.

12. The method of claim 2, wherein the anti-IgE antibody has the same properties as that produced by the cell line Accession Number BRL 10706.

13. The method of claim 2, wherein the anti-IgE antibody has the same structure as that produced by the cell line Accession Number 11131.
